(19) United States
(12) Patent Application Publication
(10) Pub. No.: US 2014/0180597 A1
(43) Pub. Date: Jun. 26, 2014

(54) EXTRACTING APERIODIC COMPONENTS FROM A TIME-SERIES WAVE DATA SET

(71) Applicant: Brigham Young University, Provo, UT (US)

(72) Inventors: Bruce Leonard Brown, Provo, UT (US); Suzanne B. Hendrix, Salt Lake City, UT (US); Dawson W. Hedges, Huntsville, UT (US)

(73) Assignee: Brigham Young Univeristy, Provo, UT (US)

(21) Appl. No.: 14/055,807

(22) Filed: Oct. 16, 2013

Related U.S. Application Data
(60) Provisional application No. 61/714,594, filed on Oct. 16, 2012.

(51) Int. Cl. A61B 5/04; G06F 17/16; G06F 17/18; A61B 5/0476

(52) U.S. Cl. CPC A61B 5/04012; A61B 5/0476; G06F 17/16; G06F 17/18
USPC 702/19; 600/544; 702/190; 702/196

(57) ABSTRACT

A method is described for extracting aperiodic components from a time-series wave data set for diagnosis purposes. The method may include collecting time-series wave data within a controlled environment were a plurality of contrasting conditions can be used in collecting the time-series wave data set. Aperiodic components can be extracted from the time-series wave data set and the aperiodic components can then be fitted to the plurality of contrasting conditions of the controlled environment to product regressed aperiodic components from which diagnostic determination can be made.

### PRIORITY DATA

This application claims the benefit of U.S. Provisional Patent Application Ser. No. 61/714,594, filed on Oct. 16, 2012, which is incorporated herein by reference.

### BACKGROUND

The term time-series may be used to refer to observations made over a period of time. For example, brain activity may be observed over time using electroencephalography (EEG, brain waves) and heart activity may be observed over time via electrocardiography (EKG, electrical activity of the heart). These observations may be represented graphically as a wave measured by a time period. For instance, an EEG wave may be represented in a line graph or line chart as a wave with peaks and valleys where the line graph has an x-axis that denotes time and a y-axis that denotes magnitude.

Diagnostic information may be derived from a time-series wave. For instance, an EEG wave can be used to distinguish an epileptic seizure from some other type of neurologic condition, or an EKG can be used to determine whether a patient is experiencing a myocardial infarction (heart attack). In some cases, a visual inspection of a line graph of a time-series wave may reveal certain characteristics within the time-series wave that provides diagnostic information. In other cases, diagnostic information may not be discernible by visually inspecting a line graph showing a time-series wave.

### BRIEF DESCRIPTION OF THE DRAWINGS

FIG. 1 is a block diagram illustrating an example system used for extracting aperiodic components from a time series wave data set for diagnostic purposes.

FIG. 2 is a block diagram illustrating another example system that can be assessed over a network and used to extract aperiodic components from a time series wave data set for diagnostic purposes.

FIG. 3 is a flow diagram that illustrates an example method for extracting aperiodic components from a time-series wave data set for classification purposes.

FIG. 4A is a graph illustrating the effects of a memory load contrasting condition for a plurality of subjects.

FIG. 4B is a graph illustrating the effects of a memory load contrasting condition for a plurality of subjects.

FIG. 4C is a graph illustrating the effects of a memory load contrasting condition for a plurality of subjects.

FIG. 4D is a graph illustrating the effects of a memory load contrasting condition for a plurality of subjects.

FIG. 5 is a structured table of graphs that illustrate aperiodic components for a single subject showing the decomposition of twelve waves into three aperiodic components.

FIG. 6 is a block diagram illustrating one example of a computing device that may be used for extracting aperiodic components from a time series wave data set for diagnostic purposes.

### DETAILED DESCRIPTION

A technology is described for extracting aperiodic components from a time-series wave data set for a variety of purposes, which can in some cases be diagnostic. As such, the present disclosure can have a wide range of potential applications. The technology can be applied to virtually any system or phenomenon that can be measured using a time-series wave. For example, in some aspects the technology can be applied to electroencephalography (EEG, brain waves), electrocardiography (EKG, electrical activity of the heart), as well as to a wide range of other biologically-derived data. Additionally, the technology can be applied, without limitation, to chemical data, geological data (including oil exploration), data derived from mechanical devices such as gasoline engines, steam engines, jet engines, and diverse motors, and the like.

In one example configuration in the realm of psychology, contrasting conditions (e.g., cogitative test components, performance test components, etc.) that may be known or hypothesized to have an effect on subject performance can be varied while recording and averaging resulting time-series waves. The time-series wave data gathered can be decomposed into latent components. Using spectral decomposition, aperiodic components can be extracted for each subject individually from the subject's set of averaged wave contours. In one aspect of the technology, the aperiodic components may be referred to as RASD (regressed aperiodic spectral decomposition) components. The RASD components and their associated multiplicative coefficients for each contrasting condition can contain diagnostic value. That is, the RASD components can be essentially “fitted” to each contrasting condition and therefore provide component wave contours that capture the process waveform created by each contrasting condition within each individual subject. Wave contours of the contrasting conditions can be unique to each individual subject, and can contain diagnostic information for the individual subject when generated and analyzed.

In one example method, aperiodic components for a subject can be extracted from a time-series wave data set and analyzed for a variety of purposes, some of which can be diagnostic. A time-series wave data set may be collected within a controlled environment where the controlled conditions include a plurality of contrasting conditions (e.g., test conditions). Component analysis of the time-series wave data can then be performed to extract aperiodic components from the time-series wave data set. The aperiodic components can represent a plurality of contrasting conditions of the controlled environment. The process can be repeated for each subject included in the time-series wave data set. Regression analysis can then be performed for each of the aperiodic components producing regressed aperiodic spectral decomposition (RASD) components. From the RASD components, relationships to classifications can be identified from among the subjects included in a given time-series wave data set. For example, based on a relationship to a classification (i.e., a feature of a RASD component that can be linked to a classification), a determination can be made that a person associated with the RASD component may suffer from a mental condition classification, such as depression. Additional non-limiting classifications can include depression, migraines, addiction, obsessive-compulsive behavior disorder, academic performance, mood disorder, schizophrenia, personality disorder, bipolar disorder, Asperger's syndrome, autism, attention deficit hyperactivity disorder (ADHD), neurosis, paranoia, incipient Alzheimer's disease, incipient Parkinson's disease, incipient heart attack, and the like.

With the understanding that the technology is not limited to EEG data, the method in its application to EEG data will be demonstrated throughout this disclosure, and more particularly, a specific type of well-controlled EEG data known as event related potentials (ERPs). An ERP is a stereotyped electrophysiological response to a stimulus, or in other words, an ERP is a measured brain response to a specific sensory, cognitive or motor event.

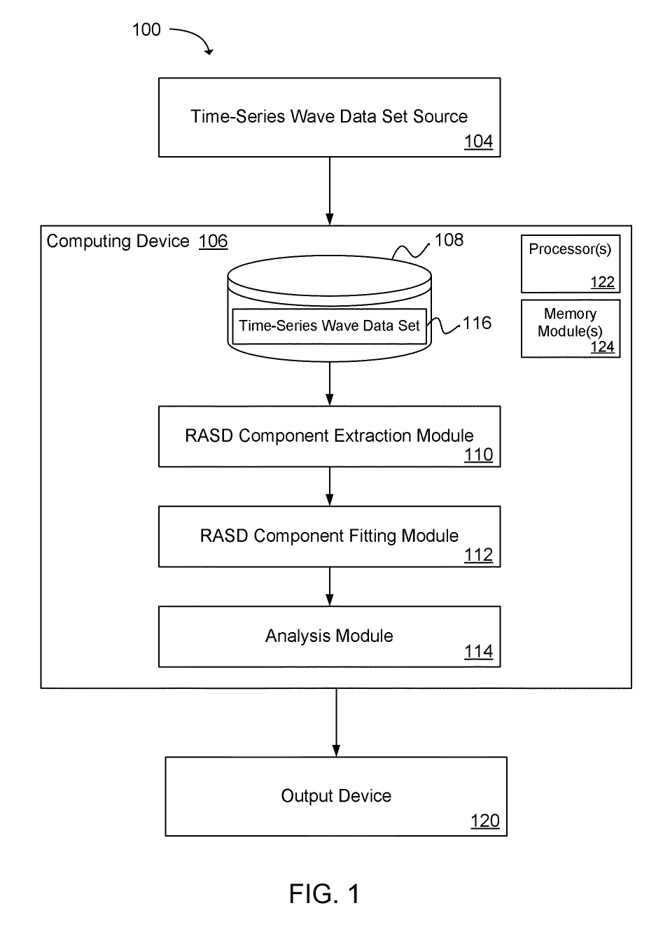

FIG. 1 is a diagram illustrating a high level example of a system 100 for extracting aperiodic components from a time-series wave data set, in some cases for diagnostic purposes. The system 100 can include a time-series wave data set source 104 (e.g., EEG recording device, ECG recording device, sound recording device, seismic monitor, etc.), a computing device 106 and an output device 120, such as a display. The computing device 106 can contain a data store 108 in which a time-series wave data set 116 can be stored. For example, the computing device 106 can receive a data set 116 from the time-series wave data set source 104 and store the data set in the data store 108.

The computing device 106 can include a number of modules that can be used to extract various components from a data set and analyze the components for characteristics that can be used to identify various conditions/characteristics of a subject from which the data set was obtained. The computing device 106 can include an RASD (regressed aperiodic spectral decomposition) component extraction module 110, an RASD component fitting module 112, an analysis module 114 as well as other services, processes, systems, engines, or functionality not discussed in detail herein.

In one example, the RASD component extraction module 110 can be used to extract a first set of RASD (reduced aperiodic spectral decomposition) components from a time-series wave data set 116. Each of the first set of RASD components extracted from the time-series wave data set 116 can represent contrasting conditions that may have been used, or may have been present when collecting the time-series wave data set 116. Contrasting conditions can be tests that may be performed while collecting time-series wave data. Examples of these tests can include cognitive tests that can be performed by a subject while collecting time-series wave data (e.g., EEG data), physical tests that can be performed by a subject while collecting time-series wave data (e.g., ECG data), performance tests that can be administered while collecting time-series wave data from a machine or engine, as well as other tests. Moreover, contrasting conditions can be conditions within a controlled environment in which the time-series wave data is being collected.

As an illustration where EEG data is collected from several subjects while cognitive tests are administered and used as contrasting conditions, the RASD component extraction module 110 can extract a first set of RASD components from the EEG data for an individual subject. In a case where the cognitive tests include a memory load component, a presence/absence component, and a replications component, for example, components can be represented in one of the extracted RASD components (i.e., a memory load RASD component, a presence/absence RASD component and a replications RASD component). It is noted for this and other applications of the present technology that the time-series wave data set can be processed as the data is collected or it can be processed following data collection. The data can be recently collected, or in some cases, the data can be retrieved from a storage collection of data and subsequently processed.

Once the first set of RASD components have been extracted from the time-series wave data set 116 where each contrasting condition may be represented by a RASD component, the first set of RASD components can be provided to the RASD component fitting module 112. The RASD component fitting module 112 can be used to fit RASD components representing other subjects included in the time-series wave data set to the results of the RASD components extracted from the time-series wave data set for a single individual. In other words, the RASD component extraction module 110 can be used to isolate wave contours within an individual (e.g., person or machine) and then the RASD component fitting module 112 can be used to identify classifications between individuals (e.g., persons or machines). The fitting process produces a second set of RASD components that can then be analyzed to determine relationships to classifications.

The second set of RASD components can then be provided to the analysis module 114. The analysis module 114 can be used to identify relationships to classifications associated with the second set of RASD components. For example, a classification such as gender can be determined by analyzing EEG RASD components. Further, using EEG RASD components, examples of classifications such as addiction, depression, obsessive-compulsive behavior, academic performance, and the like, can be made. In one example, analysis of variance (ANOVA) can be used to identify relationships to classifications. In another example, multivariate analysis of variance (MANOVA) can be used to identify relationships to classifications. As will be appreciated, various methods can be used to analyze the second set of RASD components and any method that can be used are within the scope of the technology.

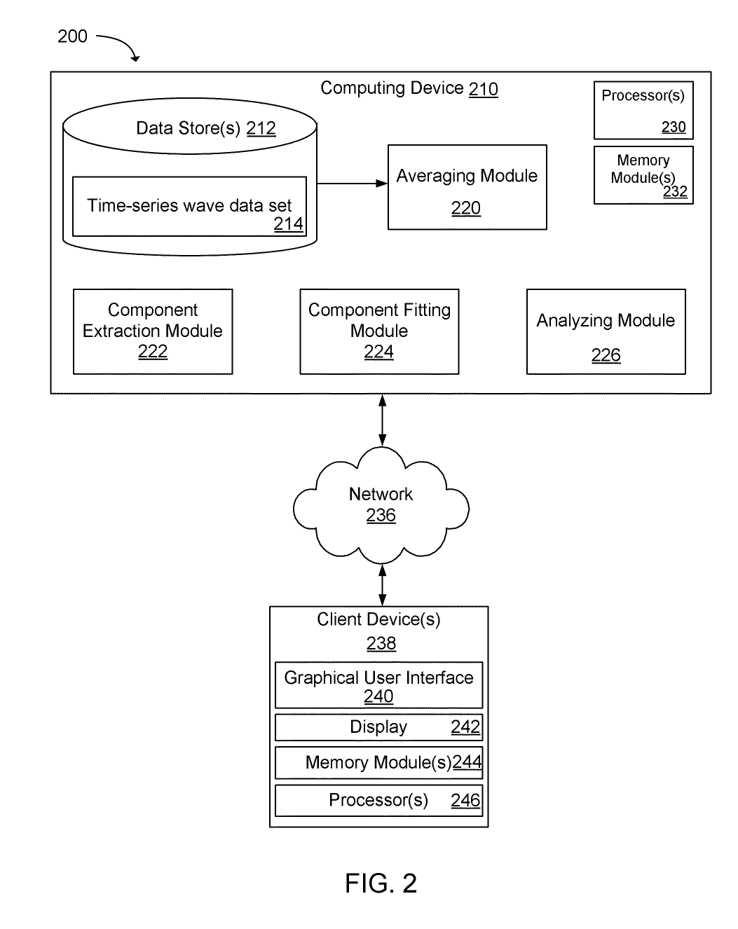

FIG. 2 illustrates an example of various components of a remote system 200 on which the present technology may be executed. The remote system can include a computing device 210 and is in communication with a client device 238 by way of a communications network 236. In one example configuration, the computing device 210 can include a data store 212, a averaging module 220, a component extraction module 222, a component fitting module 224, an analyzing module 226 as well as other services, processes, systems, engines, or functionality not discussed in detail herein.

Similar to the system described in FIG. 1, the system 200 can be used to extract aperiodic components from a time-series wave data set, in some cases for classification purposes. The data store 212 can include one or more time-series wave data sets 214 containing time-series wave data. In one example configuration, the averaging module 220 may retrieve the time-series wave data set 214 from the data store 212 and calculate an average value for selected time points of the time-series wave data set 214. For example, adjacent data values within the time-series wave data set 214 can be averaged thereby reducing the number of time points contained in the time-series wave data set 214. By averaging adjacent values within the time-series wave data set 214, the size of the time-series wave data set 214 can be reduced making the time-series wave data set 214 more manageable for extracting aperiodic components from the time-series wave data set 214. It is also contemplated that non-adjacent data values can be averaged. Furthermore, other techniques of reducing the size of the data set are also within the present scope.

The time-series wave data set 214 can then be provided to the component extraction module 222. In one example configuration, the component extraction module 222 can be used to, for example, factor the time-series wave data set 214. The time-series wave data set 214 can be factored so that the resulting factored data set contains sufficient factors to account for a majority of variances that may be contained in the time-series wave data set. An example of a time-series wave data set 214 may be a data matrix for a single subject (i.e., person or machine) where each column of the data matrix represents a time point within an event related potential (ERP) contour. The data matrix may be positive semi-definite in form where the data matrix has a rank that is less than the order of the data matrix. Moreover, the data matrix may be an arm correlation matrix of time points, a covariance matrix of time points or an SSCP (Sums of Squares and Cross Products) matrix of time points.

The component extraction module 222 can be used to identify ERP contours that represent contrasting conditions used in capturing the time-series wave data set. For example, when capturing EEG data from a subject, the subject may be asked to perform various tasks designed to measure cognitive activity. A cognitive task performed by the subject can be a contrasting condition used to capture the time-series wave data set. One example of a contrasting condition used to capture a time-series wave data set can be a memory load component. As an illustration, a subject may be fitted with EEG electrodes that are connected to an EEG recording device. The subject may be asked to remember a given set of digits (e.g., 5 and 7). The number of digits in the set (e.g., two) that a subject is asked to remember can be a memory load contrasting condition. Digits may then be shown to the subject and the subject may be instructed to press a button each time one of the digits in the set of digits is displayed. Recognizing the presence and absence of a digit as one of the digits that the subject has memorized can be another contrasting condition, namely, a presence/absence contrasting condition. The component extraction module 222 can identify ERP contours for each contrasting condition (i.e., the memory load contrasting condition and the presence/absence contrasting condition) from the time-series wave data set. It should be noted that in the above example contrasting conditions are used to demonstrate and explain the method and therefore do not limit the scope of the present technology. Any contrasting condition can be used as a component when capturing a time-series wave data set.

In an example where an arm correlation matrix of time points is used, factor analysis can be performed to extract aperiodic components from the arm correlation matrix. In an example where a covariance matrix of time points is used, principal component analysis can be performed to extract aperiodic components from the covariance matrix. And in an example where an SSCP (Sums of Squares and Cross Products) matrix of time points is used, spectral decomposition analysis can be performed to extract aperiodic spectral decomposition (ASD) components from the SSCP matrix.

In an example configuration using an SSCP matrix of time points, the component extraction module 222 can be used to extract a first set of aperiodic components by creating a matrix of time points for an individual subject. Spectral decomposition can be used to extract eigenvectors from the SSCP matrix of time points. The extracted eigenvectors capture the one or more contrasting conditions used to collect a time-series wave data set.

A latent variable scores matrix can then be created by multiplying the SSCP matrix of time points by a matrix of normalized eigenvectors derived from the extracted eigenvectors. The latent variable scores are coefficients that can be multiplied by the normalized eigenvectors matrix to obtain individual aperiodic components.

The first set of aperiodic components can be further manipulated to obtain more precise aperiodic components. That is, regression analysis can be performed on the first set of aperiodic components. Having extracted a first set of aperiodic components that represent the contrasting condition used to collect the time-series wave data set 214, the first set of aperiodic components can then be provided to the component fitting module 224. The component fitting module 224 can be used to “fit” the time-series wave data set 214 of other subjects to that of the first set of aperiodic components. By doing so, a second set of aperiodic components can be produced from the first set of aperiodic components that can be used to identify relationships to classifications associated with the second set of aperiodic components (e.g., depression, addiction, etc.).

In one example configuration, the component fitting module 224 can be used to perform component analysis on each of the aperiodic components in the first set of aperiodic components in turn. Regression analysis can then be performed using the first set of aperiodic components producing a second set of aperiodic components. The second set of aperiodic components can represent the plurality of subjects from which the time-series wave data set 214 was obtained. The second set of aperiodic components can then be provided to the analyzing module 226, and can be used to determine relationships to classifications associated with the second set of aperiodic components. In other words, the second set of aperiodic components can be used to differentiate groups of subjects. For example, where the aperiodic component of a subject may represent EEG data for a person, the aperiodic component may specify the gender of the subject, or whether the subject has depression, migraines, addiction or some type of neurologic disorder.

The results of the analysis can be provided to a user via a client device 238 and a user interface. The client device 238 can include any device that may be capable of sending and receiving data over a network 236. A client device 238 can comprise, for example, a processor-based system such as a computing device. Such a computing device can contain one or more processors 246, one or more memory modules 244, and a graphical user interface 240. A client device 238 can be a device such as, but not limited to, a desktop computer, laptop or notebook computer, tablet computer, mainframe computer system, handheld computer, workstation, network computer, or other devices with like capability. The client device 238 can include a display 242, such as a liquid crystal display (LCD) screen, gas plasma-based flat panel display, LCD projector, cathode ray tube (CRT), or other types of display devices, etc.

The various processes and/or other functionality contained on the computing device 210 can be executed on one or more processors 230 that are in communication with one or more memory modules 232 according to various examples. The computing device 210 can comprise, for example, a server or any other system providing computing capability. Alternatively, a number of computing devices 210 can be employed that are arranged, for example, in one or more server banks or computer banks or other arrangements. For purposes of convenience, the computing device 210 is referred to in the singular. However, it is understood that a plurality of computing devices 210 may be employed in the various arrangements as described above.

Various data may be stored in a data store 212 that is accessible to the computing device 210. The term “data store” refers to any device or combination of devices capable of storing, accessing, organizing and/or retrieving data, which may include any combination and number of data servers, relational databases, object oriented databases, cloud storage systems, data storage devices, data warehouses, flat files and data storage configuration in any centralized, distributed, or clustered environment. The storage system components of the data store 212 can include storage systems such as a SAN (Storage Area Network), cloud storage network, volatile or non-volatile RAM, optical media, or hard-drive type media. The data store 212 can be representative of a plurality of data stores 212, as can be appreciated.

The network 236 can include any useful computing network, including an intranet, the Internet, a local area network, a wide area network, a wireless data network, or any other such network or combination thereof. Components utilized for such a system can depend at least in part upon the type of network and/or environment selected. Communication over the network may be enabled by wired or wireless connections and combinations thereof.

FIG. 1 and FIG. 2 illustrate that certain processing modules may be discussed in connection with this technology and these processing modules may be implemented as computing services. In one example configuration, a module may be considered a service with one or more processes executing on a server or other computer hardware. Such services may be centrally hosted functionality or a service application that may receive requests and provide output to other services or consumer devices. For example, modules providing services may be considered on-demand computing that are hosted in a server, cloud, grid or cluster computing system. An application program interface (API) may be provided for each module to enable a second module to send requests to and receive output from the first module. Such APIs may also allow third parties to interface with the module and make requests and receive output from the modules. While FIG. 1 and FIG. 2 illustrate examples of systems that may implement the techniques above, many other similar or different environments are possible. The example environments discussed and illustrated above are merely representative and not limiting.

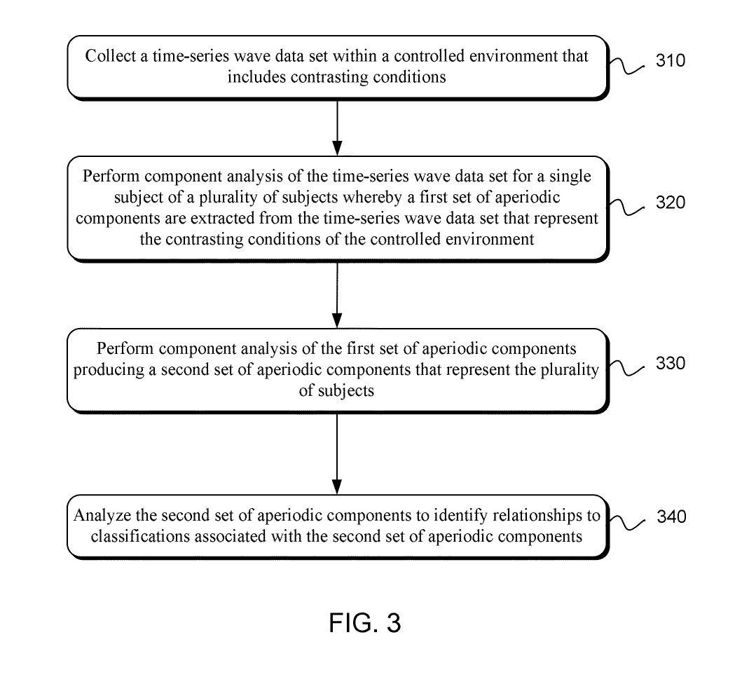

FIG. 3 is a flow diagram illustrating one example of a method for extracting aperiodic components from a time-series wave data set for classification purposes. Beginning in block 310, a time-series wave data set can be collected, in some cases within a controlled environment, which includes contrasting conditions. A controlled environment can be an environment such as a clinical lab where testing of a subject can be performed. When testing a subject, contrasting conditions can be used to collect the time-series wave data set. Contrasting conditions can be test components, such as cognitive test components, physical test components, or mechanical test components that are included in a test that a subject performs while wave data for the subject is recorded. Also, contrasting conditions can be environmental conditions that can be manipulated within the controlled environment. It is noted that for this description, as well as throughout the present disclosure, the term “subject” can refer to a living organism such as a mammal, non-mammal, lab animal, human, etc., as well as non-living items such as motors, engines, geologic formations, and the like. A subject can additionally be a space, such as the dimensions of a room used to perform acoustic measurements therein. As such, when a subject is described as “performing,” such can also include performance. A horse, for example, can be analyzed while running. As another example, an engine's performance can be analyzed in a similar manner to that described herein. The acoustics of an area having a measureable aperiodic component can also be similarly analyzed.

Returning to FIG. 3, as in block 320, component analysis of the time-series wave data set for a single subject of a plurality of subjects can be performed, whereby a first set of aperiodic components are extracted from the time-series wave data set that represent the contrasting conditions (e.g., test components) of the controlled environment. In other words, the time-series wave data set can contain wave data for a plurality of subjects (i.e., a plurality of persons or a plurality of machines). A sub-set of the time-series wave data representing a single subject of the plurality of subjects can be selected and component analysis can then be performed on the sub-set of time-series wave data resulting in a first set of aperiodic components. In one example, the above process can be repeated for each remaining subject of the plurality of subjects.

In one example configuration, an arm correlation matrix of time points from the time-series wave data set can be created and factor analysis can be used to extract aperiodic components from the arm correlation matrix. In another example configuration, a covariance matrix of time points from the time-series wave data set can be created and principal component analysis can be used to extract aperiodic components from the covariance matrix. And yet in another example configuration, an SSCP (sums of squares and cross products) matrix of time points can be created from the time-series wave data set and spectral decomposition analysis can be used to extract aperiodic spectral decomposition (ASD) components from the SSCP matrix.

After extracting the first set of aperiodic components that represent contrasting conditions, as in block 330, component analysis of the first set of aperiodic components can be performed producing a second set of aperiodic components that represent the plurality of subjects. As in block 340, the second set of aperiodic components can be analyzed to identify relationships to classifications associated with the second set of aperiodic components. For example, between subjects analysis can be performed using the second set of aperiodic components. Between subjects analysis may determine relationships contained in the second set of aperiodic components that can be tied to classifications. For example, where the time-series wave data set may contain EEG data, relationships contained in the second set of aperiodic components may be tied to cognitive classifications such as depression, migraine headaches, addiction, obsessive-compulsive disorder, and/or low academic performance.

The following provides a specific example of a method for performing the technology where the example method can be used to analyze event related potentials (ERP) of EEG wave data. ERPs produce a highly controlled and simplified wave that isolates a time-series contour of the brain processes associated with a perceptual or cognitive task from an ongoing complex combination of other brain processes. ERPs can do this by averaging recordings of brain activity (e.g., with each contour being about 750 msec. long) that are time-locked with the stimulus, such that all of the processing activity initiated by the stimulus is amplified, while unrelated ongoing brain activity is averaged out.

It should be noted that some traditional approaches to the analysis of ERPs utilize the amplitudes and the latencies of “peak-picked” components (N200, P300, LN, LP, etc.) as dependent variables to capture wave contours resulting from controlled condition manipulations. Initial results from such an approach have been disappointing, with marginally significant F ratios for each controlled condition. One possible explanation for the marginal results can be that the measured amplitudes and latencies of peaks of the wave are separated from the holistic context of the wave contour and therefore do not capture sufficient information from the wave contour to be useful for diagnostic purposes. Also, the amplitudes and latencies from the peak-picking process may not properly separate the nomothetic and ideographic information in the wave contours.

One example of deconstructing the ERP contours into separate cognitive components can involve a process of separating complex ideographic information, which can be highly specific like a fingerprint, from the simple and systematic nomothetic information produced by the contrasting conditions. In other words, spectral decomposition of the set of ERP wave contours, calculated, can be effective in separating the ideographic information in the waves from the nomothetic information. The process can be analogous to Fast Fourier Transforms (FFT) in the acoustic analysis of sound waves, in which the complex wave is decomposed into its constituent sine waves by a mathematical process. The difference may be that whereas the sine wave components in FFT are periodic and regular (i.e., consistently cyclic sine waves of a particular frequency), components extracted by an aperiodic spectral decomposition process are aperiodic and irregular in shape.

Aperiodic spectral decomposition components can reduce an error term of a nomothetic part of contrasting condition (i.e., cognitive tests) information, resulting in large F ratios. As such, the present technology could be used to extract highly valuable diagnostic information from the precise temporal microstructure of ERP data.

In one specific example, EEG wave data for seven subject persons (four males and three females) is analyzed. Subject persons are asked to remember a given set of digits, such as “two and seven,” or “eight, three, five, and nine.” Each person can then be given a series of singly presented digits on a visual display device and instructed to press a button each time one of the previously indicated digits appeared (presence responding), or to press the button each time a digit other than one of those indicated appeared (absence responding).

In a case where each subject responds 600 times, 50 times for each of six contrasting conditions, three levels of memory load (ML) for each of the two response conditions (i.e., presence responding and absence responding). The fifty waves for each contrasting condition can be averaged to create an ERP contour for each of the twelve contrasting conditions. This can be done for each of the seven subjects, and for recordings at each of five electrode locations: Fz, Cz, Oz, T3, and T4, according to the international-10-20 system. A computing device can collect simultaneously a person's reaction-time and ERP data.

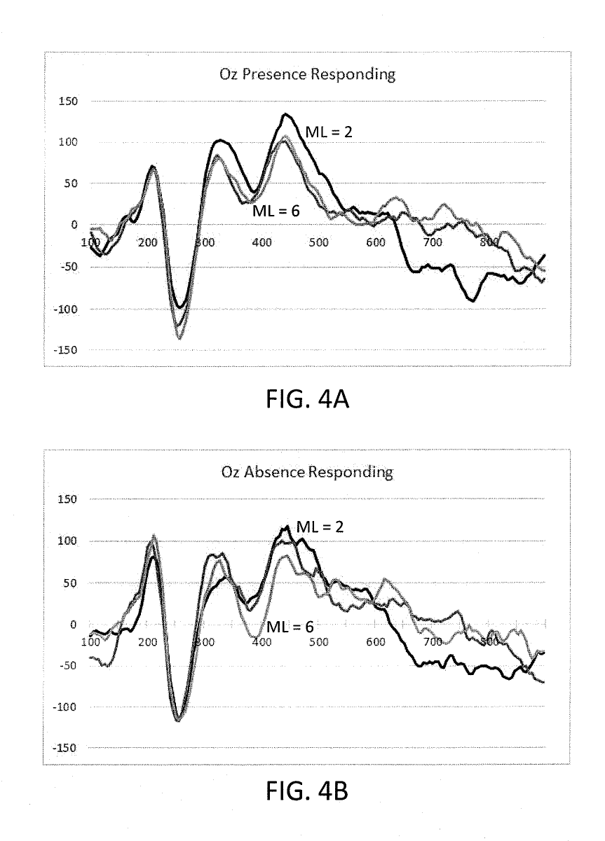

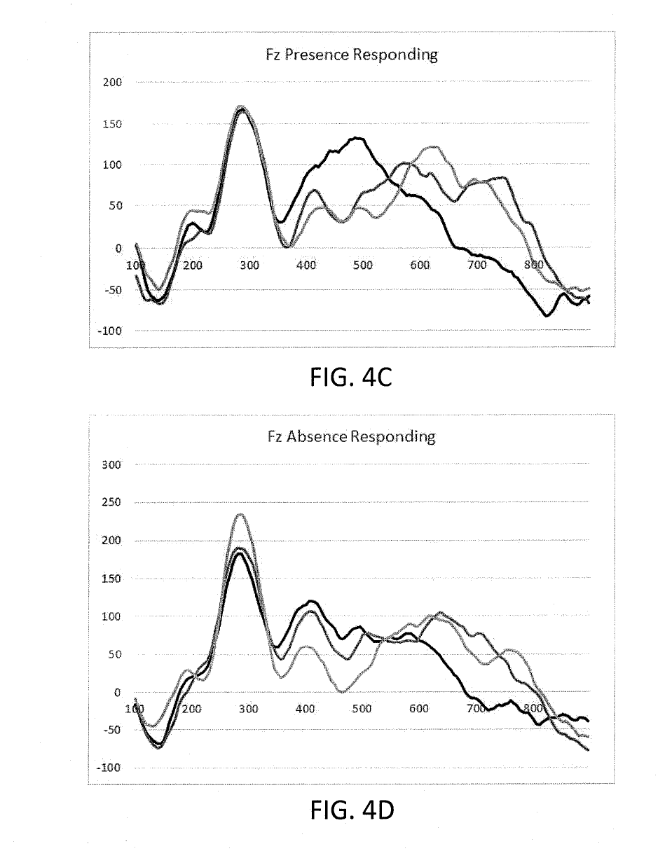

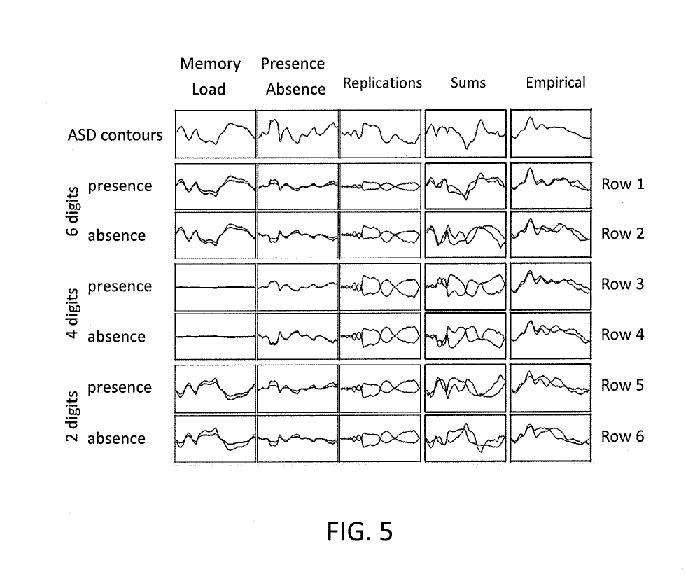

Average contours can be calculated for memory load levels of ML=2, ML=4, and ML=6, both for presence responding and also for absence responding (i.e., 6 in all), for each of the seven subjects at each of the five locations (i.e., 210 in all). Average contours for the six contrasting conditions at five locations (i.e., 30) can also be calculated by averaging the contours across the seven subjects. FIGS. 4A_D show twelve of these contours, with three ML level contours in each of the four panels. Specifically, FIGS. 4A-D provide grand averages over seven subjects showing the effects of memory load (ML=2, 4 or 6 digits). FIGS. 4A and 4B show the effects of ML of grand averages over seven subjects showing the effects of memory load (ML=2, 4 or 6 digits). FIGS. 4A and 4B show the effects of ML for presence responding, the Oz location in FIG. 4A and the Fz location in FIG. 4B. FIGS. 4C and 4D show the same information for the absence responding condition r presence responding, the Oz location in FIG. 4C and the Fz location in FIG. 4D.

Despite the effects of the contrasting conditions, the aperiodic components as confirmed by statistical tests and visual inspection reveal that the aperiodic components do not capture cognitive information with the systematic precision needed to differentiate between persons. Therefore, a method that rotates the aperiodic components can be used producing RASD (regressed aperiodic spectral decomposition) components. The method may have similarities to regressed principal component analysis, but can be applied to individual SSCP (sums of squares and cross products) matrices and covariance matrices of the present aperiodic component process.

The process can create systematic patterns in the RASD structured tables of graphs and in accompanying RASD Riemannian sphere graphs. RASD analysis can separate nomothetic information (i.e., contrasting conditions reflected in coefficients) from ideographic information (i.e., personal characteristics of each person, reflected in the RASD contours).

As an example, focusing upon single subjects, individual data matrixes for each subject having 12 rows (i.e., 3 levels of ML by 2 levels of PA by 2 replications) and 160 columns (i.e., the 160 time points in the ERP contour) can be constructed. An ASD (aperiodic spectral decomposition) analysis can comprise first creating a 160×160 SSCP matrix, covariance matrix, or correlation matrix of time points for each subject. The matrix can be positive semi-definite in form having a rank less than the matrix's order. In this example, the maximum rank of the matrix is 12, since only 12 rows go into its computation. A spectral decomposition algorithm can then be used to extract three eigenvectors from the SSCP matrix, which capture the ERP contours representing the memory load (ML) cognitive process, the presence/absence (PA) cognitive process, and a time change component from replication 1 to replication 2.

A 12×3 matrix of latent variable scores (for the ML, PA, and replication contrasting conditions) can be created by multiplying the 12×160 matrix by the 160×3 matrix of normalized eigenvectors. These latent variable scores can be the coefficients by which one multiplies the eigenvectors (the APC contours at the top of FIG. 5) to obtain the individual aperiodic wave components in rows 1 through 6 of FIG. 5. The first three columns of FIG. 5 show the individual aperiodic wave components for the ML, PA, and replications manipulations, respectively. The fourth column of wave contours in FIG. 5 is the composite sum of the wave components to the left of it, and the fifth and last column of FIG. 5 contains the actual empirical waves for each of the 12 experimental conditions. Generally, FIG. 5 shows an ASD structured table of graphs for a single subject showing the decomposition of twelve waves into three ASD components: memory load, presence versus absence responding and replications.

The process of wave contour decomposition can be accomplished by extracting a set of principal components large enough to account for nearly all variances in the original input data wave contours, and then in turn regressing the contrasting condition weights onto these principal components (i.e., the memory search data with 12 contrasting condition contours). As a result, ASD analysis and graphs (i.e., aperiodic spectral decomposition components) can be replaced by RASD analysis and graphs (i.e., regressed aperiodic spectral decomposition components).

The RASD analysis and graphs can then be provided to a “between subjects analysis” process that can extract diagnostic information from the individual RASD components. Namely, the process can be used to quantify and diagnose neuropsychiatric abnormalities from the shapes of the RASD components. The process may be similar to that used to create the RASD components described above. The process can be applied once for each RASD component (i.e., the ML component, the PA component, and the replication component) resulting in a second set of RASD components. From this second set of RASD components, “between person analyses” can be performed.

The following is a more specific example of the computational method by which RASD graphs and analyses can be created. It should be noted that multiple methods may be available that can produce similar graphical and statistical results and these methods are within the scope of this disclosure. The following is merely one method that can be used to extract aperiodic components from a time-series wave data set for diagnostic purposes.

The method may include two computational modules where the first computational module may perform “within a subject analysis” and the second computational module may perform “between subjects analysis” The first computational module can begin with a time-series wave data set containing time-series wave data for a plurality of subjects (i.e., persons) and locations (i.e., brain locations). In this example, the time-series wave data set can be a matrix of 420 rows (i.e., 12 contrasting condition contours multiplied by 35 persons/locations) and 160 columns (i.e., time data points spaced 4 msec. apart that can define each wave contour).

A 12×160 sub-matrix for one subject at one EEG location can be isolated for initial analysis. The 12×160 matrix can be reduced to a 12×80 matrix by averaging adjacent data points, or alternatively, the entire 12×160 matrix can be analyzed. For simplicity of illustration, this example will use the 12×80 matrix.

Next, an 80×80 correlation matrix can be created from the 12×80 matrix, from which principal component analysis can be used to extract 9 components (enough to account for nearly all variances) in a 80×9 factor loadings matrix and a 12×9 factor scores matrix.

A 12×3 contrasting conditions matrix (with levels of 2, 4, or 6 for Memory Load; 1 or 2 for Presence/Absence; and 1 or 2 for Replications) is constructed, standardized, and then appended to the 12×9 factor scores matrix.

Each of the contrasting conditions (i.e., the Memory Load condition, the Presence/Absence condition, and the Replications condition) can be regressed onto the 9 principal components to create a 9×3 regression coefficients matrix.

The 80×9 factor loadings matrix can be post-multiplied by the 9×3 regression coefficients matrix to obtain an 80×3 regressed factor loadings matrix. The three columns of the 80×3 regressed factor loadings matrix are wave contours representing the three contrasting conditions memory load, presence/absence, and replications. Similarly, the 12×9 factor scores matrix can be post-multiplied by the 9×3 regression coefficients matrix to obtain a 12×3 regressed factor scores matrix.

The process of the first computational module can be repeated for all 35 combinations of subjects and locations. The 35 regressed factor loadings matrices, each being an 80×3 matrix, can be appended to one another to create an 80×105 regressed factor score input matrix that can be provided to the second computational module.

Moving now to the second computation module, the 80×105 regressed factor score input matrix is input into the second computation module where an 80×35 contrasting condition sub-matrix containing one of the contrasting conditions is isolated for the first analysis. In a case were the memory load contrasting condition (ML) is first selected, an 80×80 correlation matrix can be created from the 80×35 ML sub-matrix, and principal component analysis can be used to extract enough components to account for nearly all variances in the 80×80 correlation matrix. An 80×21 factor loadings matrix and a 35×21 factor scores matrix (with the 35 rows representing the seven persons at each of 5 EEG locations) are collected from the principal component analysis.

In a case were six design contrasts are used for analysis, namely, gender of the person and a binary contrast for each of five EEG locations (i.e., CZ, FZ, OZ, T3, and T4), a 35×6 design contrasts matrix is standardized and adjoined to the 35×21 factor scores matrix.

Each of the 6 design contrasts (i.e., gender, CZ, FZ, OZ, T3, and T4) can be regressed onto the 21 principal components resulting in a 21×6 regression coefficients matrix. The 80×21 factor loadings matrix can then be post-multiplied by the 21×6 regression coefficients matrix to obtain an 80×6 regressed factor loadings matrix. Similarly, the 35×21 factor scores matrix can be post-multiplied by the 21×6 regression coefficients matrix to obtain a 35×6 regressed factor scores matrix. In the case where the focus of the analysis is to identify gender differences, a 35×1 vectors of factor scores can be isolated.

The process above can be repeated with the presence/absence contrasting condition and the replications contrasting condition as the input to the analysis, and combine the three 35×1 vectors of factor scores into a 35×3 regressed factor scores matrix. An ANOVA (analysis of variance) or a MANOVA (multivariate analysis of variance) can then be used to compare the factor scores of men and women.

A MANOVA on these data yields a Wilks' lambda value of 0.0245, which corresponds to a multivariate R-squared value of 0.9755. Each of the univariate ANOVAs also indicates a strong and significant relationship, with memory load being the strongest (F(1,33)=527.69, p<0.0001, R2=0.941), presence/absence responding next strongest (F(1,33)=373.93, p<0.0001, R2=0.919), and replications the least strong (F(1,33)=334.87, p<0.0001, R2=0.910).

Although the focus of the second computational module is the regressed factor scores that are used to differentiate groups of people, the regressed factor loadings may also be of use. For example, a vector plot sphere can be useful in interpreting the meaning of the location in which each group and person may be located, as can the envelope plots that show with temporal precision the contrast in wave contours between the differentiated groups.

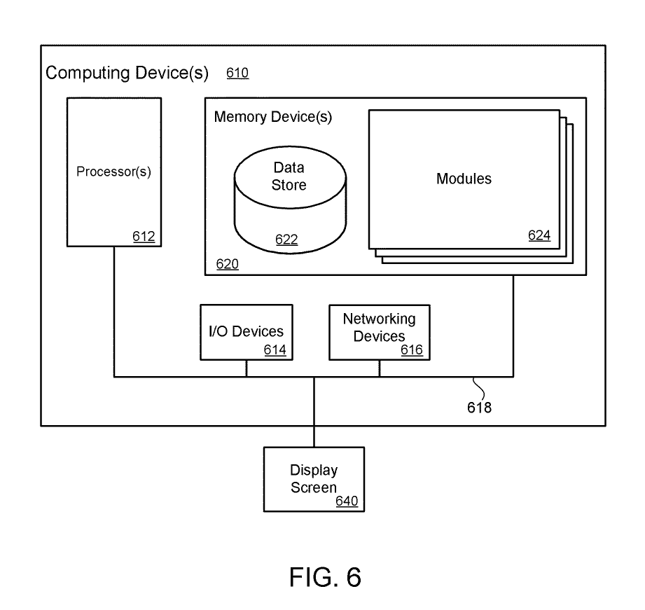

FIG. 6 illustrates one non-limiting example of a computing device 610 on which modules of this technology may execute. A computing device 610 is illustrated on which a high level example of the technology may be executed. The computing device 610 may include one or more processors 612 that are in communication with memory devices 620. The computing device 610 may include a local communication interface 618 for the components in the computing device. For example, the local communication interface may be a local data bus and/or any related address or control busses as may be desired.

The memory device 620 may contain modules 624 that are executable by the processor(s) 612 and data for the modules 624. The modules 624 may execute the functions described earlier. A data store 622 may also be located in the memory device 620 for storing data related to the modules and other applications along with an operating system that is executable by the processor(s) 612.

Other applications may also be stored in the memory device 620 and may be executable by the processor(s) 612. Components or modules discussed in this description that may be implemented in the form of software using high programming level languages that are compiled, interpreted or executed using a hybrid of the methods.

The computing device may also have access to I/O (input/output) devices 614 that are usable by the computing devices. An example of an I/O device is a display screen 640 that is available to display output from the computing devices. Other known I/O device may be used with the computing device as desired. Networking devices 616 and similar communication devices may be included in the computing device. The networking devices 616 may be wired or wireless networking devices that connect to the internet, a LAN, WAN, or other computing network.

The components or modules that are shown as being stored in the memory device 620 may be executed by the processor(s) 612. The term “executable” may mean a program file that is in a form that may be executed by a processor 612. For example, a program in a higher level language may be compiled into machine code in a format that may be loaded into a random access portion of the memory device 620 and executed by the processor 612, or source code may be loaded by another executable program and interpreted to generate instructions in a random access portion of the memory to be executed by a processor. The executable program may be stored in any portion or component of the memory device 620. For example, the memory device 620 may be random access memory (RAM), read only memory (ROM), flash memory, a solid state drive, memory card, a hard drive, optical disk, floppy disk, magnetic tape, or any other memory components.

The processor 612 may represent multiple processors and the memory 620 may represent multiple memory units that operate in parallel to the processing circuits. This may provide parallel processing channels for the processes and data in the system. The local interface 618 may be used as a network to facilitate communication between any of the multiple processors and multiple memories. The local interface 618 may use additional systems designed for coordinating communication such as load balancing, bulk data transfer and similar systems.

While the flowcharts presented for this technology may imply a specific order of execution, the order of execution may differ from what is illustrated. For example, the order of two more blocks may be rearranged relative to the order shown. Further, two or more blocks shown in succession may be executed in parallel or with partial parallelization. In some configurations, one or more blocks shown in the flow chart may be omitted or skipped. Any number of counters, state variables, warning semaphores, or messages might be added to the logical flow for purposes of enhanced utility, accounting, performance, measurement, troubleshooting or for similar reasons.

Some of the functional units described in this specification have been labeled as modules, in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of executable code may, for instance, comprise one or more blocks of computer instructions, which may be organized as an object, procedure, or function. Nevertheless, the executables of an identified module need not be physically located together, but may comprise disparate instructions stored in different locations which comprise the module and achieve the stated purpose for the module when joined logically together.

Indeed, a module of executable code may be a single instruction, or many instructions and may even be distributed over several different code segments, among different programs and across several memory devices. Similarly, operational data may be identified and illustrated herein within modules and may be embodied in any suitable form and organized within any suitable type of data structure. The operational data may be collected as a single data set, or may be distributed over different locations including over different storage devices. The modules may be passive or active, including agents operable to perform desired functions.

The technology described here may also be stored on a computer readable storage medium that includes volatile and non-volatile, removable and non-removable media implemented with any technology for the storage of information such as computer readable instructions, data structures, program modules, or other data. Computer readable storage media include, but is not limited to, RAM, ROM, EEPROM, flash memory or other memory technology, CD-ROM, digital versatile disks (DVD) or other optical storage, magnetic cassettes, magnetic tapes, magnetic disk storage or other magnetic storage devices, or any other computer storage medium which may be used to store the desired information and described technology.

The devices described herein may also contain communication connections or networking apparatus and networking connections that allow the devices to communicate with other devices. Communication connections are an example of communication media. Communication media typically embodies computer readable instructions, data structures, program modules and other data in a modulated data signal such as a carrier wave or other transport mechanism and includes any information delivery media. A “modulated data signal” means a signal that has one or more of its characteristics set or changed in such a manner as to encode information in the signal. By way of example and not limitation, communication media includes wired media such as a wired network or direct-wired connection and wireless media such as acoustic, radio frequency, infrared and other wireless media. The term computer readable media as used herein includes communication media.

Reference was made to the examples illustrated in the drawings and specific language was used herein to describe the same. It will nevertheless be understood that no limitation of the scope of the technology is thereby intended. Alterations and further modifications of the features illustrated herein and additional applications of the examples as illustrated herein are to be considered within the scope of the description.

Furthermore, the described features, structures, or characteristics may be combined in any suitable manner in one or more examples. In the preceding description, numerous specific details were provided, such as examples of various configurations to provide a thorough understanding of examples of the described technology. It will be recognized, however, that the technology may be practiced without one or more of the specific details, or with other methods, components, devices, etc. In other instances, well-known structures or operations are not shown or described in detail to avoid obscuring aspects of the technology.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements may be devised without departing from the spirit and scope of the described technology.

What is claimed is:

1. A method for extracting aperiodic components from a time-series wave data set for classification purposes, comprising:
under control of one or more computer systems configured with executable instructions, collecting the time-series wave data set that includes contrasting conditions;
performing component analysis of the time-series wave data set for a single subject of a plurality of subjects whereby a first set of aperiodic components are extracted from the time-series wave data set that represent the contrasting conditions;
performing component analysis of the first set of aperiodic components producing a second set of aperiodic components that represent classifications of conditions associated with the plurality of subjects; and
analyzing the second set of aperiodic components to identify relationships to classifications associated with the second set of aperiodic components.

2. A method as in claim 1, wherein the contrasting conditions further comprise components of a cognitive task performed by a person.

3. A method as in claim 1, further comprising creating a correlation matrix of time points from the time-series wave data set and performing factor analysis to extract aperiodic components from the arm correlation matrix.

4. A method as in claim 1, further comprising creating a covariance matrix of time points from the time-series wave data set and performing principal component analysis to extract aperiodic components from the covariance matrix.

5. A method as in claim 1, further comprising creating an SSCP (Sums of Squares and Cross Products) matrix of time points from the time-series wave data set and performing spectral decomposition analysis to extract aperiodic spectral decomposition (ASD) components from the SSCP matrix.

6. A claim as in claim 1, further comprising calculating an average value for selected time points of the time-series wave data set.

7. A claim as in claim 1, wherein gender is a classification associated with the second set of aperiodic components used to identify relationships within the second set of aperiodic components.

8. A claim as in claim 1, wherein identifying relationships to classifications associated with the second set of aperiodic components further comprises identifying relationships to classifications from the group consisting of depression, migraines, addiction, obsessive-compulsive behavior disorder, academic performance, mood disorder, schizophrenia, personality disorder, bipolar disorder, Asperger's syndrome, autism, attention deficit hyperactivity disorder (ADHD), neurosis, paranoia, incipient Alzheimer's disease, incipient Parkinson's disease and incipient heart attack.

9. A claim as in claim 1, further comprising using analysis of variance (ANOVA) to identify relationships to classifications associated with the second set of aperiodic components.

10. A claim as in claim 1, further comprising using multivariate analysis of variance (MANOVA) to identify relationships to classifications associated with the second set of aperiodic components.

11. A claim as in claim 1, further comprising selecting from the group consisting of discriminant analysis, logistic regression analysis, multiple regression analysis, canonical correlation analysis and signal detection theory (SDT) analysis to identify relationships to classifications associated with the second set of aperiodic components.

12. A claim as in claim 1, wherein the time-series wave data set is collected within a controlled environment.

13. A computer implemented method, comprising:
under control of one or more computer systems configured with executable instructions, collecting time-series wave data that includes a plurality of contrasting conditions;
extracting an ASD (aperiodic spectral decomposition) component from the time-series wave data using spectral decomposition; and
fitting the ASD component to the plurality of contrasting conditions thereby providing an RASD (regressed aperiodic spectral decomposition) component from which diagnostic determinations are made.

14. A claim as in claim 13, wherein collecting time-series wave data further comprises collecting electroencephalography (EEG) data.

15. A claim as in claim 14, wherein time-series wave data is collected from an electrode placed to capture EEG data from a specified brain location.

16. A claim as in claim 13, further comprising providing a graphical representation of a plurality of RASD components in a structured graph.

17. A claim as in claim 13, further comprising providing a graphical representation of a plurality of RASD components within a Riemannian sphere graph.

18. A claim as in claim 13, further comprising providing a graphical representation of a plurality of RASD component factor scores within a RASD coefficient scatterplot graph.

19. A claim as in claim 13, wherein time-series wave data is collected under controlled conditions.

20. A non-transitory machine readable storage medium, including program code, when executed to cause a machine to perform the method of claim 12.

21. A system for extracting aperiodic components from a time-series wave data set, comprising:
a processor;
a memory device including instructions that, when executed by the processor, cause the processor to execute:
a factoring module to perform component analysis of a time-series wave data set where principal components are extracted from the time-series wave data set that represent a plurality of factors used to collect the time-series wave data set;
a regression module to create regressed principal components by performing regression analysis of the principal components; and
an analysis module to analyze the regressed principal components to identify characteristics associated with the regressed principal components.

22. A system as in claim 21, further comprising an averaging module to calculate an average value for selected time points of the time-series wave data set.
